(19) United States
(12) Patent Application Publication
(10) Pub. No.: US 2021/0354370 A1
(43) Pub. Date: Nov. 18, 2021

(54) MATERIAL SYSTEM AND METHOD FOR PRODUCING PATIENT-SPECIFIC COMFORT EAR ADAPTERS FOR ACOUSTIC HEARING, AUDIO, AND HEARING PROTECTION INDUSTRY

(71) Applicant: Fraunhofer-Gesellschaft zur Foerderung der angewandten Forschung e.V., Muenchen (DE)

(72) Inventors: Herbert Wolter, Wuerzburg (DE); Somchith Nique, Wuerzburg (DE); Johannes Schwaiger, Wuerzburg (DE); Carina Kolb, Wuerzburg (DE); Helma Baeuerlein, Wuerzburg (DE)

(73) Assignee: Fraunhofer-Gesellschaft zur Foerderung der angewandten Forschung e.V., Muenchen (DE)

(21) Appl. No.: 17/260,351

(22) PCT Filed: Jul. 17, 2019
(86) PCT No.: PCT/EP2019/069205

§ 371 Date: Jul. 21, 2021

(30) Foreign Application Priority Data
Jul. 20, 2018 (DE) 102018117631.7

(51) Int. Cl. B29C 64/124; B33Y 70/10; B33Y 10/00; B33Y 80/00; C08G 75/045

(52) U.S. Cl. CPC B29C 64/124; B33Y 70/10; B33Y 30/00; B33Y 80/00; C08G 75/045; B33Y 10/00

(57) ABSTRACT

The invention relates to a shaped body and a process for its production by means of a radiation-induced printing process, characterized in that the shaped body is produced from a liquid or viscous material which contains a starting component with cycloolefinic groups, a thiol with at least two thiol groups per molecule and an initiator and/or catalyst for the light-induced thiol-ene addition reaction between the thiol groups and a double bond of the cycloolefinic groups, and the solidification takes place by directing light from a radiation source onto a region of a surface of a substrate, a layer of the liquid or viscous material located there being subjected to organic polymerisation by the action of radiation and thereby solidified, whereupon further layers of the liquid or viscous material, which are each located on the layer of the most recently solidified material, are successively solidified with the aid of this radiation source.

The present invention relates to a process for producing a shaped body by means of a 3D printing technique, in which a liquid or viscous material containing cycloolefinic groups and thiol groups is subjected to organic cross-linking and thus solidification by the action of light. The invention further relates to a shaped body with special thermomechanical properties which is obtainable by this process and can be used in particular as an earmould.

Currently, there are approximately 5.3 million hearing aids in use in the Federal Republic of Germany, and the trend in sales is rising. In 2014, this was already over 1 million in Germany alone. The individual earmoulds for behind-the-ear hearing aids (BTE hearing aids) or in-the-ear hearing aids (ITE hearing aids), which sit in the auricle and in the auditory canal and connect the hearing aid to the ear, are called earmoulds. Various materials are used to produce hearing aids, whereby these can be divided into hard and soft earmoulds depending on the material. As a standard, earmoulds are manufactured on the basis of acrylates, especially methyl methacrylate. These are mostly two-component curing materials for hard earmoulds. The disadvantage here is the residual monomer content in the cured material, which can lead to severe allergic reactions. In addition, hard earmoulds tend to produce annoying whistling noises, especially in the case of severe hearing loss, which are caused by the feedback of sound when the earpiece does not sit firmly in the ear and does not lie against the skin all around. Soft earmoulds, on the other hand, seal better, reducing the risk of feedback. Silicone is used as a permanently elastic material for this purpose. However, the insertion of soft earmoulds is more difficult due to the blunt surface.

Furthermore, the first thermoformable materials are available which are hard at room temperature and soft/flexible at body temperature and therefore adapt well to the individual ear shape. The earmould, which is firm and dimensionally stable at room temperature, is therefore easy to insert and adapts to the individual ear shape at body temperature, ensuring a high level of wearing comfort. However, the disadvantages here are the toxic fumes during processing of the material and the purely organic, monomer-based cross-linking. Another major disadvantage of all the material systems mentioned so far is the costly, conventional production process of the earmoulds. This can be done by a so-called positive-negative-positive process. In this process, the ear is first moulded with silicone rubber, then a negative mould is made with silicone, agar-agar or plaster, which is finally filled with the appropriate material. In an elaborate finishing step, the blanks have to be shaped and painted manually with hand-held milling and grinding tools. The high material loss due to subtractive manufacturing and the necessary negative mould is unfavourable. Another disadvantage is that special tools have to be used, which is associated with additional costs due to tool wear, as well as the costly/time-intensive manual work. Another process uses an automated CAD/CAM milling process. Here, the impression of the ear is scanned and its 3D data is processed and modelled with software before the earmould is milled from a prefabricated milling disc (a so-called blank). This is followed by another polishing and lacquering step. Here, too, there is a high material loss and tool wear.

These disadvantages of the currently used production process of earmoulds can be avoided by manufacturing them using 3D printing processes. The positive impression of the ear form is digitally recorded by means of a 3D scanner. The virtual data of the ear impression, processed with special CAD programs, can then be converted directly into the actual final product using a 3D printer. The automated manufacturing process eliminates many manual steps, thus reducing the amount of work involved. Even complicated geometries can be formed. With 3D printing processes, material loss is also low.

However, the commercial materials described above are not suitable for use in the printing process. For 3D printing of earmoulds, one-component resin systems based on (meth)acrylate or silicones are used. However, the purely organic polymers have the disadvantage that they are not monomer-free and therefore not anti-allergenic. Moreover, up to now only hard earmoulds can be made from these materials, which are not very flexible and do not adapt well to the ear. Until now, it has been possible to coat the earmould for allergy sufferers (e.g. with glazing or gold plating) in order to avoid direct contact of the plastic with the ear canal.

As already mentioned, earmoulds made of silicone are difficult to insert into the ear because of their soft surface.

The invention is based on the problem of providing shaped bodies, including in particular earmoulds, which show a slight softening at rising temperatures and thus malleability, so that they can be used e.g. for patient-specific final components with adapted, high-quality property profile for hearing acoustics/audio and hearing protection and other purposes. These shaped parts should be made of a material that can be formed into the desired shapes using 3D printing as mentioned above.

In particular, it would be desirable to be able to produce an earmould whose materials—unlike those known up to now—are both biocompatible (and in particular anti-allergenic) and have a thermo effect for optimal adaptation to the ear at body temperature and thus a high degree of wearing comfort.

In addition, colourless/transparent versions as well as different colour variants should be available for them, if possible, for attractive aesthetics. Thus, it should be possible to create transparent colourless, transparent coloured, opaque colourless and opaque coloured shaped bodies, whereby the colours can be different. An important colour variant is skin-coloured.

The problem is solved by providing a process for producing a shaped body by means of a radiation-induced printing process, characterized in that
the shaped body is produced by solidification of a liquid or viscous material which is a starting component with cycloolefinic groups, preferably contains a silicic acid (hetero)polycondensate modified with such groups, a thiol with at least two thiol groups per molecule and an initiator and/or catalyst for the light-induced thiol-ene-addition reaction between the thiol groups and a double bond of the cycloolefinic groups, and
the solidification is effected by directing light from a radiation source onto a region of a surface of a substrate, a layer of the liquid or viscous material located there being subjected to organic polymerisation by the action of radiation and thereby solidified, whereupon further layers of the liquid or viscous material, which are each located on the layer of the most recently solidified material, are successively solidified with the aid of this radiation source.

The problem is further solved by providing a shaped body of a material formed by photo-induced thiol-ene-addition of a thiol compound having at least two thiol groups per molecule to a starting component having cycloolefinic groups, wherein the shaped body suffers a drop in its memory module E of at least 500 MPa, preferably at least 800 MPa, even more preferably at least 1200 MPa, within a temperature window of −25° C. to 110° C., preferably of −15° C. to 70° C., more preferably of −15° C. to 60° C., at a temperature rise of at most 20 K. Preferably it has a memory module E_{RT} (at 22° C.) of between 20 and 4000 MPa, more preferably between 500 and 2600 MPa, a minimum memory module E′ₘᵢₙ of between 5 and 300 MPa, preferably between 15 and 60 MPa, and a temperature T_{W} (at 2·E′ₘᵢₙ) of between 0° C. and 110° C., more preferably between 10° C. and 60° C. All these values are determined in each case by a DMA test of the shaped part using NETZSCH DMA242C with the following settings: Distance between supports=20 mm; frequency=1 Hz; heating rate=2.0° C./min; max. dyn. force=4.00 N; atmosphere=air.

More precisely, the present invention concerns the following points [1] to [15]

[1] A process for producing a shaped body with the aid of a radiation-induced printing process, characterized in that
the shaped body is produced by solidification of a liquid or viscous material which contains a starting component with cycloolefinic groups, a thiol with at least two thiol groups per molecule and an initiator and/or catalyst for the radiation-induced thiol-ene-addition reaction between the thiol groups and a double bond of the cycloolefinic groups, and
the solidification is effected by directing light from a radiation source onto a region of a surface of a substrate, a layer of the liquid or viscous material located there being subjected to organic polymerisation by the action of radiation and thereby solidified, whereupon further layers of the liquid or viscous material, which are each located on the layer of the most recently solidified material, are successively solidified with the aid of this radiation source,

wherein the shaped body undergoes a drop in its memory module of at least 500 MPa within a temperature window of −25° C. to 110° C. at a temperature rise of at most 20 K, determined by a DMA test on the shaped body by means of NETZSCH DMA242C with the following settings: support distance=20 mm; frequency=1 Hz; heating rate=2.0° C./min; max. dyn. force=4.00 N; atmosphere=air.

Preferably, the mentioned drop of the memory module is at least 800 MPa.

[2] The process according to point [1], wherein the liquid or viscous material is in a bath container with a bottom which is at least partially transparent, and the substrate is a platform immersed in the liquid or viscous material and movable away from the bottom of the bath container.

In points [1] and [2] a process is preferred in which the irradiation is carried out with a wavelength in the range of less than 500 nm, preferably at 380 to 420 nm, and/or in which the layer thickness of the individual layers is selected in the range of 3 to 150 μm, preferably 25 to 100 μm, and/or in which irradiation lasts 0.2 to 100 s, preferably 2 to 40 s, and/or in which the bath material is exposed with an intensity of 4 to 50 W/m², preferably 4 to 10 W/m².

[3] The process according to any of the preceding points, wherein the starting component with cycloolefinic groups is a silicic acid (hetero)polycondensate modified with mono- or bicycloolefinic groups, preferably norbornenyl groups.

[4] The process according to one of the above points, wherein the starting component with cycloolefinic groups is a silicic acid (hetero)polycondensate of or with silanes in which radicals bonded to the silicon via carbon have one norbornenyl group, one norbornenyl group and one hydroxy group or two norbornenyl groups, or wherein the starting component with cycloolefinic groups is a purely organic compound which has two terminal norbornenyl groups.

[5] The process according to any of the preceding points, wherein the thiol having at least two thiol groups per molecule is a trithiol or a mixture of two trithiols.

A combination of the features of points [1], [3] and [5] is particularly preferred. A combination of the features of points [1], [4] and [5] is even more preferred.

[6] The process according to point [5], wherein the trithiol is trimethylolpropanetri(3-mercaptopropionate) (TMPMP) and/or 2,3-di((2-mercaptoethyl)-thiol-1-propanethiol (DMPT).

In the process according to any of the preceding points, it is preferred that the ratio of thiol groups to cycloolefinic or bicycloolefinic groups is 0.5-1.2 to 1.0 and in particular about 0.9 to 1.0.

According to any of the preceding points, a process is preferred wherein a phosphine oxide, preferably a diphenylphosphine oxide and most preferably 2,4,6-trimethylbenzoyldiphenylphosphine oxide (LTPO) is added to the liquid or viscous material as an initiator and/or catalyst for the light-induced thiol-ene addition reaction.

[7] The process according to one of the preceding points, in which the shaped body is produced in a bath of the liquid or viscous material and, after its formation, is removed from the bath, washed with a solvent, dried and then post-cured, the post-curing preferably being photoinitiated.

[8] The process according to any of the preceding points, wherein the liquid or viscous material further contains a thiol-ene stabilizer, in particular pyrogallol.

According to one of the preceding points, a process is preferred in which the liquid or viscous material is a composite containing a particulate and/or fibrous additive in addition to the starting component with cycloolefinic groups, the thiol and the initiator and/or catalyst.

According to any of the preceding points, a process is preferred in which the particulate additive is inorganic particles, preferably those of titanium dioxide, zirconium dioxide, zinc oxide, zinc sulphide, silica or glass or a combination of several of the said materials, zirconium dioxide being particularly preferred.

According to one of the preceding points, a process is preferred wherein the inorganic particles have an average primary particle diameter of 1 to 100 nm, preferably of 5 to 30 nm, and are present in agglomerated or dispersed form.

According to any of the preceding points, a process is preferred wherein the inorganic particles are provided with a coating modified with an organically polymerizable residue such that this organically polymerizable residue is subjected to a thiol-ene addition reaction upon irradiation of the liquid or viscous material.

According to any of the preceding points, a process is preferred in which the liquid or viscous material further comprises a dissolved or particulate material capable of absorbing light of the exposure wavelength.

According to one of the preceding points, a process is preferred in which the said material is a dissolved fluorescent material.

[9] The process according to any of the preceding points, wherein the shaped body is suitable for at least one use from the group comprising use as an earmould, use for hearing acoustics/audio purposes, use for hearing protection purposes, use as an insertion needle, stent, infusion needle and use as a carrier for cultivating biological organisms or cells.

[10] A shaped body obtainable by the process of any of the preceding claims. The body according to claim [10] comprises the reaction product formed by the photoinduced thiol-ene-addition reaction of a thiol compound having at least two thiol groups per molecule with a starting component having cycloolefinic groups according to claim [1].

Within a temperature window of −25° C. to 110° C. and at a temperature rise of no more than 20 K, the shaped body according to point [10] will suffer a drop in its memory module of at least 500 MPa, preferably at least 800 MPa, determined by a DMA test of the shaped body using NETZSCH DMA242C with the following settings: support distance=20 mm; frequency=1 Hz; heating rate=2.0° C./min; max. dyn. force=4.00 N; atmosphere=air.

[11] The shaped body according to point [10], having been formed of individual layers with a thickness in the range of 3 to 150 μm in a spatial direction or in which the layers have been formed by continuous exposure to light.

[12] The shaped bodies according to point [10] or [11] with a memory module E_{RT} (at 22° C.) between 20 and 4000 MPa, a minimum memory module E′ₘᵢₙ between 5 and 300 MPa, preferably between 15 and 60 MPa, and a temperature T_{W} (at 2·E′ₘᵢₙ) between 0° C. and 110° C., in each case determined by a DMA test according to the process specified in point [1].

Preferably, the shaped part according to one of the points [10] to [12] additionally contains a filler.

According to any of the preceding points, a process is preferred wherein the filler consists of or contains dispersed zirconia particles with a primary particle diameter of about 5-50 nm, the zirconia particles preferably being norbornenyl and/or methacrylate functionalized.

Preferably, the shaped body according to one of the points [10] to [12] has a translucency of at least 75%, preferably at least 78%.

[13] The shaped body according to one of the points [10] to [12], which is an earmould, an injection needle, a stent, or an infusion needle.

[14] The use of a shaped body according to any of points [10] to [13] in the medical field, in particular selected from the use as an earmould and/or for use for hearing acoustics/audio and/or hearing protection purposes or as a piercing or infusion needle, stent or as a carrier for cultivating biological organisms or cells.

[15] The use of a shaped body according to one of the points [10] to [13] as an optical or mechanical switch.

As far as the shaped body has been produced according to the process according to the invention, it is usually composed of layers of a thickness of 10 to 150 μm.

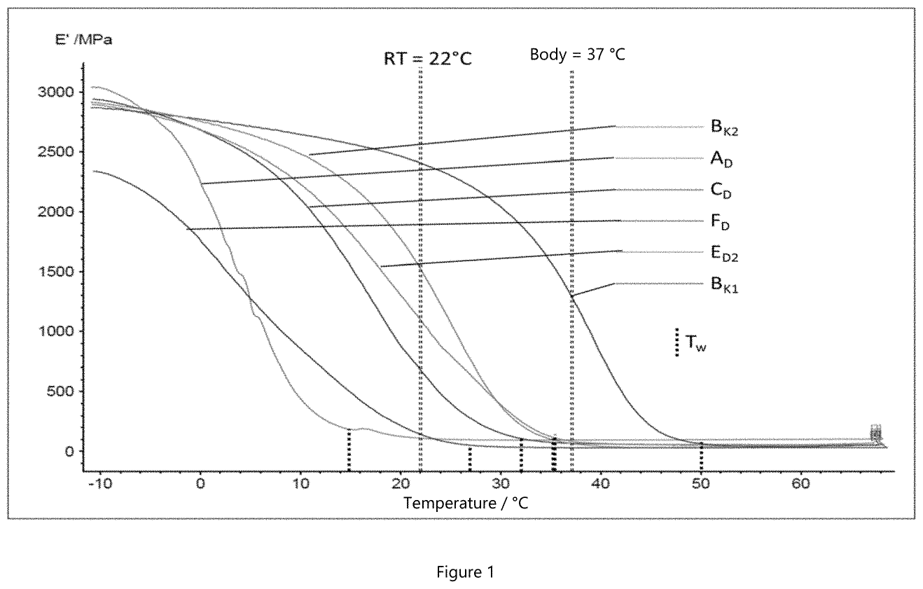

FIG. 1 shows a diagram with the DMA curves of some selected shaped bodies of different composition and production/post-curing processes.

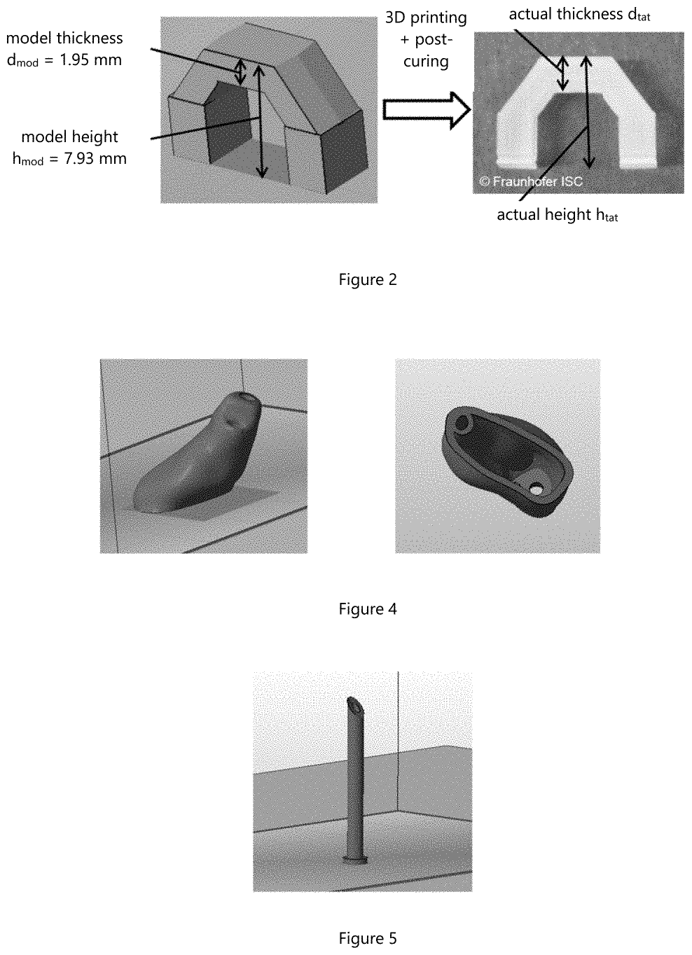

FIG. 2 shows a test specimen produced according to the process of the invention, which is suitable for the determination of defective polymerization.

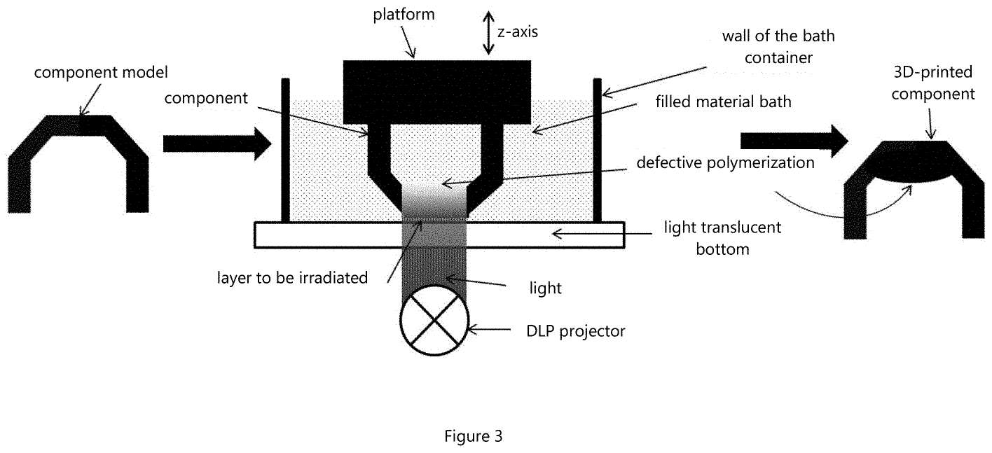

FIG. 3 illustrates the pressure of a test specimen and thus a possible principle of the process according to the invention.

FIG. 4 shows the model for an earmould (exterior view/interior view).

FIG. 5 shows the model of an infusion needle (with continuous internal opening).

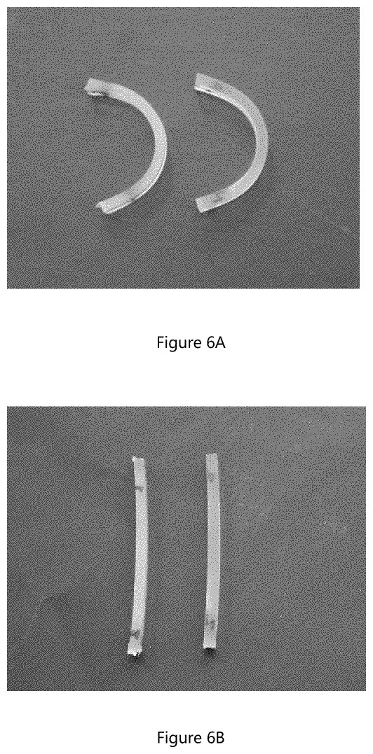

FIG. 6A shows deformed test specimens after heating and clamping in a mould.

FIG. 6B shows the test specimens that have returned to their original shape.

The present invention uses a process in which the body is produced by solidification of a liquid or viscous material, the solidification being effected by directing light from a radiation source onto a region of a surface of a substrate on which there is a layer of the liquid or viscous material, which layer is subjected to organic polymerisation by the action of radiation from this radiation source and is thereby solidified, whereupon further layers of the liquid or viscous material, each of which is located on the layer of the most recently solidified material, are successively solidified by means of this radiation source.

The “light from a radiation source”, as defined in the present case, need not necessarily be in the visible range. UV light, for example, is included in this term.

Layers that are “on the layer of the most recently solidified material” are located on this layer when viewed in the direction of the radiation source. If, for example, the substrate is placed in a bath and the substrate surface is irradiated through a bottom of the bath container which is transparent to the light of the radiation source, the layers of the liquid or viscous material to be solidified in each case are located below the substrate together with the layers already solidified on it.

According to the invention, all techniques can be used which start from a liquid or viscous (“pasty”), i.e. flowable material which can be solidified locally by exposure to light. Each layer is exposed flat, i.e. usually with a fixed amount in z-direction and with any outline of the respective layer in x-y-direction. The respective area(s) of each layer can be exposed simultaneously (in this case, the exposure is a two-dimensional exposure), or it/they is/are scanned with a beam (the entire area is then exposed in narrow strips rather than simultaneously). These techniques are called “printing techniques”; they belong to the group of 3D printing techniques.

According to the invention, the so-called DLP (Digital Light Processing) is particularly suitable for the production of the shaped bodies. This is a material-saving, automated and thus fast and cost-effective process in which the material is located inside a bath, i.e. in a bath container. Here, a substrate in the form of a platform, which can be moved in the z-direction, is placed in the bath in such a way that between the bottom of the bath container and the downwardly facing surface of the platform or the already solidified layers there is a thin layer of the bath material in each case, which can be exposed and solidified, whereupon the platform is moved and in the process is finally pulled upwards by a layer thickness in the z-direction, so that bath material flows into the resulting gap. Each solidified layer usually has a constant thickness in the z-direction, but this may be infinitesimally small, as explained in more detail below. In the x-y direction, however, it can have any shape (also multi-part) or outline, so that the resulting shaped body can also have e.g. undercuts or individual columns. It is also possible to form several individual shaped bodies at the same time. Solidification at the bottom of the bath is achieved by irradiating the bath material through the translucent bottom (or a translucent part thereof) or a translucent wall of the bath container. Either a writing beam (e.g. in stereolithography) can be used so that individual areas of the surface are “scanned”, i.e. exposed in narrow strips one after the other, or the exposure can be carried out all at once (simultaneously) over the entire surface, which is thus exposed simultaneously. The latter is preferred because the area exposure is much faster. The preferred exposure wavelengths are those with which a single-photon polymerization reaction can be effected. Exemplary sketches explaining the exposure path can be found in FIG. 3.

The DLP printer used may have a vibration system that allows periodic excitation of the material tray. Frequencies of preferably 100 Hz to 40 kHz are possible. The excitation can, for example, be pulsating or in interval mode. This reduces the viscosity when using non-Newtonian material systems. These systems can be particularly useful for composites with higher filler content.

The DLP printer used in the present invention may additionally have a modified tub system with translational displacement capability, e.g. a tub with several chambers, for example a three-chamber tub. A multi-chamber tub can be used for the alternating use of different printing materials or the use of a tub for washing the components before changing the material.

The process according to the invention can then have the following characteristics:

A process of producing a shaped body by means of a radiation-induced printing process comprising the following steps in the order given:
Layering step in a tub 1:
   providing a substrate,
   providing a flowable starting material described in the present invention,
   providing a bath wherein the substrate is immersed in the flowable starting material,
   solidifying the flowable starting material on the surface of the substrate to produce a coated substrate,
   removing of the coated substrate;
Wash step in a tub 2:
   preparing of the coated substrate,
   washing of the coated substrate, optionally with the use of a vibration system for more effective cleaning, and
   removing the washed coated substrate.

The washed coated substrate can then be used as the substrate in a further coating build-up step in tub 1. Alternatively, the washed coated substrate can be used as the substrate in a layer build-up step in tub 3, whereby the flowable starting materials in tub 3 and tub 1 differ from each other. For further layer build-up steps, additional tubs with other flowable starting materials can be used.

The cycle of layer build-up and washing step can be repeated until the desired shaped body is formed.

In a preferred configuration, the DLP printer has a vibration system and at least two trays, at least one tray being designed to be capable of performing a layering step and at least one tray being designed to be capable of performing a washing step.

In addition, a material feed system can be used that enables multi-component printing with a gradient in the z-direction. A system for tempering the printing material, preferably up to 70° C., can be used to reduce the viscosity, especially for composites with a higher filler content. For high-resolution components a pixel size of 39 to 63 μm, preferably 20 to 30 μm, can be used.

When using a single-chamber tub, a doctor blade system can be used to improve the distribution of the printing material, especially in high-viscosity systems.

The DLP printer and printing process may have the following features: Wavelength: 280 to 800 nm, preferably about 385 nm or about 405 nm; Irradiance: 0.6-40 mW/cm²; Vat Deflection Feedback System: laser sensor technology for monitoring the printing process; tub made of optical highly transparent silicone for highest precision+tub with glass/film combination for highly viscous materials; layer thicknesses: 10-300 μm; recording of the sensor measurement data; applying a substrate on the stamp possible.

A further development of the DLP process is the so-called CLIP process (Continuous Liquid Interface Production). Here, the bottom of the bath through which the radiation of the exposure source falls is preferably oxygen permeable. This prevents polymerisation in the immediate vicinity of the bath bottom (the oxygen-inhibiting layer adjacent to the bottom is usually 20-30 μm thick) and eliminates the separate and relatively time-consuming step of controlled flow of liquid/viscose material into the narrow gap, which is normally necessary with DLP and is intended for polymerisation of the next layer. The exposure can therefore be continuous while the platform is continuously pulled upwards (in z-direction). This of course has the consequence that no individual layers with a thickness measurable in z-direction are formed. Nevertheless, the process is a variant of printing in layers, since the solidification takes place simultaneously in each x-y plane. In this sense, the term “layer” should therefore, according to the invention, also include layers of infinitesimal thickness.

In general, according to the invention, printing processes are preferred where the pressure is applied within a bath of the material to be solidified. Among these, the aforementioned DLP process, including the CLIP variant, is particularly preferred. Both, the DLP process and the CLIP-version, can be combined with all other aspects of the invention listed below.

Another example of 3D printing is stereolithography (SLA) in the strict sense, where the shaped body is formed by placing a carrier just below the surface of a bath or, alternatively, near a translucent bath bottom, in such a way that only a thin layer of material lies above/below it. This is solidified (scanned) with the aid of a laser in accordance with a previously prepared digital form, whereupon the carrier is lowered/raised until the solidified layer is covered by a further liquid layer or until a further liquid layer passes between the solidified layer and the bath bottom. This layer is then also solidified with the laser beam. Each layer has a fixed thickness (thickness) in the z-direction, but can have any shape in the x-y-direction, so that a shaped body can also be produced with undercuts, for example. Further processes that can be used according to the invention are the so-called μ-stereolithography (μ-SLA), Multi Jet Modelling (MJM) and Poly Jet Printing (PJP) as well as variations of some of these processes, in which, for example, doctor blade systems are used, e.g. Lithography-based Ceramic Manufacturing (LCM), or in which, for example, film systems are used, e.g. Film Transfer Imaging (FTI) (e.g. Admaflex Technology), or further process variations, e.g. Hot Lithography (working temperature preferably up to 120° C.) or the TwoCure process or Mask Stereolithography (MSLA). For the MJM and PJP, a liquid photosensitive material is applied layer by layer to a platform via a print head, and each deposited strand (which is to be regarded as part of a layer in the sense of the invention) is immediately cured by a light source integrated e.g. in the print head (this is an example of a technique in which the respective layer is not exposed simultaneously but in narrow strips, which corresponds to the above-mentioned “scanning”). To produce overhanging structures, several print heads are used to create support structures which must be mechanically removed or washed out after printing. In the Film Transfer Imaging process, the light-sensitive material is applied to a platform via a transport foil and cured e.g. layer by layer by means of a projector. This process is especially suitable for highly viscous materials. The same applies to Film Transfer Imaging according to the Admaflex technology, in which the material to be cured is also fed via a film. The LCM, on the other hand, works with a doctor blade. The material trough rotates in a circle after each layer solidification, so that new material is applied to the bottom of the trough with the squeegee. Otherwise, there is great similarity with the DLP principle. Higher viscosity materials are also favourable for this process. The TwoCure process uses photochemical cross-linking as well as solidification with cold. Cold, waxy, but not chemically solidified material, which serves as a support, can then be liquefied at room temperature after printing. Finally, mask stereolithography is also worth mentioning, in which, as in DLP, a building platform projects into a material bath and irradiation takes place from below through the bottom of the bath, which is permeable to radiation. In contrast to DLP, however, the exposure field is not generated by a DLP chip, but by a liquid crystal exposure mask.

Processes using single-photon polymerization are preferred, since only with this process can whole layers be exposed simultaneously in the x-y-direction and thus exposed to polymerization much faster.

If DLP technology is used for the process according to the invention, the material inside the bath is solidified so that surface effects that could possibly occur can be avoided. A smaller material supply is also required compared to systems in which the building platform enters the material bath and is exposed from above. The latter technique also often requires a wiper or squeegee to produce a smooth surface.

The term “multitude of layers” means that, according to the invention, the shaped body is composed of a very large number of layers (theoretically even infinitely many, if the CLIP process is used). The actual number depends on the structure and size of the shaped body and the selected layer thickness; the layer thickness usually varies between 10 and 150 μm, but may be even less if necessary. The size of the shaped bodies will usually be in the mm or cm range, with possible deviations upwards or downwards.

The terms “shaped body” and “component” are used synonymously herein.

By “liquid or viscous material”, the invention means a material which in any case is still fluid. However, the flowability may also be relatively low.

In the present invention, the term “silicic acid (hetero)polycondensate” shall always include both silicic acid polycondensates (with or without foreign heteroelements) and silicic acid heteropolycondensates containing foreign heteroelements as mentioned.

The starting component with cycloolefinic groups to be used according to the invention can preferably be one with monocycloolefinic and/or bicycloolefinic groups.

As bicycloolefinic groups, the following can be preferred:

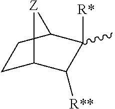

where R* is H or an organic radical, e.g. an alkyl radical having 1 to 12 carbon atoms, which may be unsubstituted or substituted by a functional group,

R** is H or an organic radical, e.g. an alkyl radical having 1 to 12 carbon atoms, which may be unsubstituted or substituted by a functional group, or wherein R** is an organic radical containing C═C groups, and

Z is —O— (i.e. an oxygen bridge), or —(CHR***)ₙ— with n=1, 2, or 3 or greater than 3 (e.g. up to 20), R*** is H, alkyl. Suitable examples are

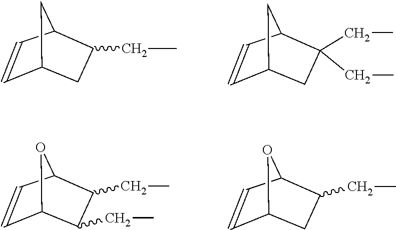

In general, the nobornenyl group is the preferred variant with respect to the bicycloolefinic group.

The monocycloolefinic groups which may be used are those of the above formula with the definitions of the radicals R* and R** given for them, in which Z is not present (the bonds to Z specified in the formula then represent bonds to hydrogen atoms).

Bicycloolefinic groups are preferred to monocycloolefinic groups.

As starting components with cycloolefinic groups, purely organic substances (compounds with at least two such groups) can be used. However, it is preferable to use a silicic acid polycondensate modified with cycloolefinic groups which has been produced from or with silanes which have at least one radical which is bonded to the silicon via carbon and which is substituted with at least one cycloolefinic group. In both cases, the preference for specific cycloolefinic radicals is as described above. In addition, the cycloolefinic groups mentioned above as preferred are also to be considered preferred in all of the individual cases below.

The cycloolefinic group can be bonded directly or via a coupling group to the carbon skeleton of a hydrocarbon-containing residue of a silicon atom. Examples of the groups and radicals through which this radical can be bonded to the silicon are disclosed inter alia in DE 196 27 198 A1; there are mentioned —(CHR⁶—CHR⁶)ₙ— with n=0 or 1, —CHR⁶—CHR⁶—S—R⁵—, —C(O)—S—R⁵—, —CHR⁶—CHR⁶—NR⁶—R⁵, —Y—C(S)—NH—R⁵, —S— R⁵—, —Y—C(O)—NH—R⁵—, —C(O)—O—R⁵—, —Y—CO—C₂H₃(COOH)—R⁵—, —Y—CO—C₂H₃(OH)—R⁵— and —C(O)—NR⁶—R⁵—, wherein R⁵ can be an alkylene, arylene, arylenealkylene or arylenealkylene in this context and R⁶ can be hydrogen, alkyl or aryl preferably having 1 to 10 carbon atoms.

It is preferred that the silica polycondensate has at least one bicycloolefinic group per —Si—O base unit. However, it is also possible to build up from or integrate silanes with or without cycloolefinic groups which carry further functional groups, e.g. polymerizable C≡C-containing groups. These can be (meth-)acrylate groups, for example, which lead to a later additive polymer structure (in the sense of homopolymerization or thiol-ene-polyaddition) and thus to a corresponding stiffening of the structure of the later shaped body. Alternatively, silanes with groups which are bonded to the silicon via carbon and which carry at least one hydroxy group or one carboxylic acid group in addition to one or two (bi)cycloolefinic groups can be used as starting materials for the silicic acid polycondensate.

The expressions “(meth)acrylic” and “(meth)acrylate” shall include respectively acrylic and acrylate, and methacryl and methacrylate.

In particular, the silicic acid (hetero)polycondensate structures can have the following formula (1)

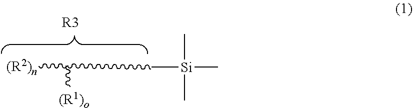

wherein the radicals and indices have the following meanings:

R¹ denotes a mono- or bicycloolefinic group. When a thiol is added, this is accessible to a thiol-ene-polyaddition and, if it is a bicycloolefinic group, it can also be polymerized by a ROMP (ring opening metathesis polymerization).

R² is selected from

(a) organically polymerisable groups that are accessible to thiol-ene-polyaddition when a thiol is added, but are not accessible to ROMP; and

(b) —OH, —COOH, carboxylic acid and other esters and their salts.

Special variants of the compounds with the formula (1) are known e.g. from DE 102011054440A1

The radical R³ is a hydrocarbon-containing radical which is bound to the silicon via carbon.

If two groups (R¹) accessible to a ROMP or one such group (R¹) as well as an organically polymerizable group (R²) are present, groups are available which can be cured gradually, possibly by different curing mechanisms and thus also at different speeds. This can be used for the production of shaped bodies with gradual mechanical and thermomechanical properties. If thiol is added to such a silica (hetero)polycondensate in a deficit, it is possible to add a ROMP catalyst (with sensitivity at a different wavelength than that of the initiator used for thiol-en addition) to the bath material. Then the shaped body produced according to the invention can be subsequently post-crosslinked either completely or spatially selectively by ROMP. This selective crosslinking in turn produces a body with different mechanical properties.

Different radicals R³ may be present in the silicic acid (hetero)polycondensates of formula (1). Such a system can be specifically and easily adjusted by the ratio of the starting materials to one another. In this way, a large number of similar condensates can be produced from a single starting material, which differ in their physical properties. Condensates with at least two groups on one residue R³, each of which is accessible to a thiol-ene addition, can be cured to an organic polymer with very dense organic cross-linking. Other curing reactions such as ROMP or the polymerization by polyaddition of residues R² with the meaning (a) (and here especially of double bonds of existing (meth-)acrylic groups) can also be finely graded, as explained in detail in the mentioned DE 10 2011054440 A1.

The silicic acid (hetero)polycondensate modified with cycloolefinic groups can be produced, for example, from silanes which have radicals bonded to the silicon via carbon atoms, each of which has one or more of the cycloolefinic groups mentioned. Hydrolysis of such silanes results in a —Si—O—Si—-based polymer structure. In addition to silicon, the silicic acid polycondensate may also contain, to a lesser extent, heteroelements such as B, Al, Zr, Sn, Zn, Ti and the like, as known from the state of the art, including the aforementioned DE 102011054440 A. These can be incorporated into the polycondensate by adding e.g. the corresponding alkoxides.

In the case of purely organic systems, at least two cycloolefinic groups are required per basic unit (i.e. in the molecule). Although this embodiment of the invention is less preferred, in cases where a purely organic matrix system is desired (matrix designates the material without any inorganic fillers which may then be added), it is advantageous over known purely organic systems in that the shaped body produced with it exhibits anti-allergenic behaviour, since it does not contain any (meth)acrylate-based monomers.

Furthermore, the liquid or viscous material to be used for the process according to the invention contains a thiol with at least two thiol groups per molecule. In this context, the term “molecule” shall not only refer to monomeric compounds but also to oligo- or polymeric compounds such as (hydrolytically produced from silanes) silicic acid (hetero)polycondensates. In any case, the molecule must contain at least two thiol groups, although in the case of condensates or the like, the number is of course usually much higher, e.g. about one thiol group per silicon atom. Thiol-containing systems based on silicic acid polycondensate are described as examples in WO 97/02272 A1 and DE 19627220 A1 on pages 30-33 of the publication. These can be used without restriction, provided they contain at least one thiol group per silane unit. Instead, purely organic systems (monomeric compounds, oligomers) can be used, which must contain at least two thiol groups per molecule. The following thiols are examples: trimethylolpropanetri(3-mercaptopropionate) (TMPMP), trimethylolpropanetri(mercaptoacetate) (TMPMA), pentaerytritoltetra(3-mercaptopropionate) (PETMP), pentaerytritol tetra(mercapto acetate) (PETMA), glycol dimercapto acetate; glycol di(3-mercaptopropionate), ethoxylated trimethylolpropanetri(3-mercaptopropionate), biphenyl-4-4′-dithiol, P-terphenyl-4,4″-dithiol; 4,4′-thiobisbezenethiol; 4,4′-dimercaptostilbene, benzene-1,3-dithiol; benzene-1,2-dithiol; benzene-1,4-dithiol, 1,2-benzene-dimethanthiol; 1,3-benzenedimethanthiol; 1,4-benzenedimethanthiol, 2,2′-(ethylenedioxy)diethanthiol; mercaptoethyl ether and 1,6-hexanedithiol; 1,8-octanedithiol; 1,9-nonanedithiol, which may be used singly or in combination. These examples already show that the large number of different thiols (e.g. with different lengths, different hydrophilicity/hydrophobicity, and different numbers of thiol groups) enables a wide range of material properties to be achieved.

Usually the thiol is used in a small deficit (e.g. 70-90 mol %) to the cycloolefinic groups, but in some cases it is advantageous to increase the proportion to 100% or even above. Then any SH groups that remain free can be used for further reactions.

A photoinitiator is also required so that the cycloolefin groups of the starting component and the thiol groups of the thiol contained in the liquid or viscous material can undergo a light-induced (radical) addition reaction. Phosphine oxides such as 2,4,6-trimethylbenzoyldiphenylphosphine oxide (LTPO) can be used for this purpose. If thermal post-curing is also planned, an additive should continue to be added to the liquid or viscous material subjected to the printing process to initiate thermal polymerization. It is also advantageous to use mixtures of the various initiators. Examples of initiators that can be used are also described in EP 3 090 722 A1 (see p. 9).

During exposure (irradiation) of the liquid or viscous material to be used for the process according to the invention, the cycloolefinic groups react with the thiols as cross-linking agents in the course of a thiol-ene-polyaddition. To stabilize the liquid or viscous material, the addition of a stabilizer (also referred to as “thiol-ene stabilizer”) may be necessary in certain cases. This is because a premature thiol-ene (addition) reaction of the components should naturally be avoided. Such stabilization is particularly necessary or at least beneficial if the system contains tightened rings, for example bicycloolefinic groups such as norbornenyl groups. Stabilizers for thiol-ene systems are known from the literature. Examples are pyrogallol, gallic acid, propyl-, octyl-, laurylgallate, 4-methoxyphenol, butylhydroxytoluene, (iso)eugenol, tocopherol, 4-tert-butylbrenzcatechin, phenothiazine, hydroquinone, 4-tert-butylphenol and others, as disclosed in Edler M. et al.: Enhancing the stability of UV-curable thiol/vinyl carbonate resins; Journal of Applied Polymer Science, 2017, Esfandiari P. et al: Efficient Stabilization of Thiol-ene Formulations in Radical Photopolymerization; Journal of Polymer Science: Part A, 2013, 51 and Hoyle, C. E. et al: Thiol-Enes: Chemistry of the Past with Promise for the future; Journal of Polymer Science: Part A, 2004, 42.

Although the starting materials and the underlying cross-linking reaction are not new, it has been shown quite surprisingly that the resulting products have previously unknown thermomechanical properties. Therefore, especially in combination with good biocompatibility (e.g. skin-friendly, anti-allergenic), they appear to be particularly suitable for use in the medical field, i.e. on the human body, and here in particular for use as inserts that can be adapted to the ear, such as earmoulds. Further application possibilities result from the fact that many of the shaped bodies produced show a drop in their memory module E′ from over 2000 MPa to only a few hundred MPa or even less in a narrow temperature range difference, e.g. of 15 K. If, as is often the case, this drop in E-modulus occurs in a temperature window between below room temperature (down to approx.—15° C.) and approximately the temperature of the human body (37° C.), the shaped bodies according to the invention are suitable for medical purposes. Thus, piercing or infusion needles in the body become soft, and the earmoulds mentioned above adapt their shape to the ear canal in which they were inserted, which greatly increases wearing comfort.

Polyaddition, i.e. curing, is achieved by irradiation during the 3D printing process used, preferably by implementing a DLP process. It is therefore possible to use a material-saving, automated and thus fast/cost-effective process to make available e.g. high-quality, patient-specific components for the hearing acoustics/audio and hearing protection sector, especially for long-term use, but not only for this sector.

When manufacturing bodies/components with overhangs or undercuts, in some cases a—literature-known—defect can occur. In the case of materials with a high translucency in the uncured and cured state, the light may not be completely absorbed by the layer to be exposed, but instead be transmitted and passed on into the already printed layers, especially in the z-direction (so-called overcuring). This is not a problem for cuboid and similar shaped bodies, as the light is only transmitted into already cured layers. For complicated shapes such as earmoulds this becomes problematic. Here, when printing layers that have a larger outline than previous ones, the light is partly guided back into the material bath, which triggers polymerisation of the material there. This causes an undesired hardening of material and thus an accumulation of material at these points. This effect is called “Defective polymerisation” (FD) or “overcuring” (see FIG. 3).

According to the invention, defective polymerization (FD) in the DLP technique, including further developments such as CLIP, is preferably determined using special test specimens that have the largest possible and easily measurable overhang and can be printed quickly. The sample bodies used are those with at least two different sections delimited from one another parallel to the z-direction, wherein one section A has at least one, preferably several, solidified layers which are formed starting from the surface of the platform, and one section B has solidified layers which do not extend to the building platform in the z-direction. The layers of section B may or may not be the most recently solidified layer or several such layers solidified in the process. They can have the form of a single, thick layer or have several, thinner layers or lamellae (e.g. webs). At least one section of the specimens must have the most recently formed layers. This can be one of the sections A or B or a combination of both; however, there may just as well be a third section C, which additionally or exclusively contains the most recently printed layers.

The components are thus designed in such a way that, after the printing process is completed, a section B of the component, viewed in the z-direction, does not extend as far as the building platform, so that a gap with uncured material (or with air if the shaped part is no longer in the bath) remains between the cured side of section B, which is “at the top” during the process, and the building platform, or that gaps with uncured material or air remain between cured parts in this section B of the component, viewed in the z-direction.

In a preferred embodiment, the test specimens have the shape of bridges or have the shape of an “H”. In both cases, the test pieces are two beams resting against the building platform (section A), with a crossbeam between the two beams which does not rest against the building platform (section B), but leaves a free space between its side “above” during the procedure and the movable platform. At the end of the procedure, when this crossbeam is in contact with the bottom of the bath, the test piece has the shape of a bridge, as shown in FIG. 1. If the crossbeam does not reach the bottom of the bath, but the beams of sections A do, the test piece has the shape of an “H”.

The dimensions of a virtual model for each specimen in the z-direction on which the process is based, including the total component height, shall be known, and the actual dimensions in the z-direction of the specimen produced by the process shall be determined after the process is completed.

After taking into account the shrinkage of the components due to post-hardening, the FD can be calculated using the following formula:

$$FD = ( \frac{{d}_{tat} \cdot {h}_{mod}}{{d}_{mod} \cdot {h}_{tat}} - 1 ) \cdot 100 = ( 4 \frac{1}{15} \cdot \frac{{d}_{tat}}{{h}_{tat}} - 1 ) \cdot 100 [ \% ]$$

wherein h_{mod} denotes the height of the virtual model of the sample body in the z-direction, hₜₐₜ denotes the specific height of the generated sample body in the z-direction, d_{mod} denotes the dimensions of the or a layer in section B of the virtual model of the sample body, and dₜₐₜ denotes the specific height of said layer in section B of the generated sample body.

Using bridges as shown in FIG. 2, it was determined in concrete terms how strongly the FD is affected as a function of the material or variable process parameters by relating the actual thickness of the crossbeam (dₜₐₜ) to that of the digital model (d_{mod}).

In order to be able to realize complex component geometries, e.g. with overhanging structures, suitable stabilizers are used as additives which absorb the radiation used for the polymerization reaction. Such stabilizers must not be confused with stabilizers that are intended to prevent premature thiol-ene addition. Herein, the radiation absorbing additives are referred to as photoabsorbers/photostabilizers or light absorbers/light stabilizers, optical brighteners, but also as UV absorbers/UV stabilizers, since a preferred process of the invention is a 3D printing process using radiation with wavelengths in the range of the transition from UV to visible (preferably in the range at about 360 to 490 nm). Of course, the absorption spectrum of the photostabilizer may have to be adapted to the wavelength of radiation used in the respective process.

Light stabilizers can be used to counteract the problems associated with overcuring and overhanging structures can be printed with dimensional accuracy. Unfilled to highly filled materials with and without light absorbers can be used and the respective effects of light absorbers and/or fillers can be determined. Depending on the requirements, light absorbers, especially inorganic and organic light absorbers and optical brighteners can be added to the compositions used according to the invention, whereby the latter have comparable properties to the light absorbers, i.e. can also reduce the FD. Organic light absorbers act e.g. via the mechanism of energy dissipation. In this respect, it is possible to refer to G. Wypych, Handbook of UV Degradation and Stabilization, ChemTec Publishing, Toronto 2015, p. 34, 43-46.

Examples of inorganic light absorbers are titanium oxide, zirconium oxide or zinc oxide. Examples of organic light absorbers are benzophenones such as DHDMBP=2,2′-dihydroxy-4,4′-dimethoxy-benzophenone, Cyasorb UV-416=2-(4-benzoyl-3-hydroxyphenoxy)ethylacrylate, Cyasorb UV-531=2-hydroxy-4-n-octoxy-benzophenone, Cyasorb UV-9=2-hydroxy-4-methoxy-benzophenone, Chiguard BP-1=2,4-dihydroxy-benzophenone, Chiguard BP-4=2-hydrox-4-methoxy-benzophenone-5-sulfonic acid, Chiguard BP-2=2,2′,4,4′-tetrahydroxy-benzophenone and benzotriazoles such as Tinuvin 327=2-(3,5-di-tert-butyl-2-hydroxyphenyl)-5-chloro-2H-benzotriazol, Chiguard 323=2-[3-(2H-benzotriazol-2-yl)-4-hydroxyphenyl]ethyl methacrylate, Chiguard R-445 (benzotriazol), Chiguard 5431=2,2′-methylene bis(6-(2H-benzotriazol-2-yl) 4-1,1,3,3, tetramethyl butyl)phenol, Tinuvin P=2-(2′-hydroxy-5′-methylphenyl)-benzotriazol, Tinuvin 326=2-(5-chloro-2H-benzotriazol-2-yl)-6-(1,1-dimethylethyl)-4-methyl-phenol, Tinuvin 328=2-(2H-benzotriazol-2-yl)-4,6-ditertpentyl-phenol, Chiguard 5411=2-(2′-hydroxy-5′-tert-octylphenyl)-benzotriazol, Chiguard 234=2-[2-hydroxy-3,5-di-(1,1-dimethylbenzyl)]-2H-benzotriazol. Examples of optical brighteners are TBT=2,5-thiophenediylbis(5-tert-butyl-1,3-benzoxazol), Uvitex OB-ONE=2,2′-(1,2-ethylenediyldi-4,1-phenylene)bisbenzoxazol, Eutex 127=1,1′-biphenyl-4,4′-bis[2-(methoxyphenyl)ethenyl], Eutex KCB=2,2′-(1,4-naphthalenediyl)bis-benzoxazole, Eutex CBS=1,1′-biphenyl-4,4′-bis[2-(sulphophenyl)ethenyl]disodium salt, Eutex KSN=4,4-bis (5-methyl-2-benzoxazol)-ethylene or tris(dibenzoylmethane) mono(1,10-phenanthroline)europium(II).

Organic light absorbers which have reactive groups which can co-polymerise with reactive groups of the starting compounds used, e.g. (meth)acrylate groups, are particularly preferred. Examples are Chiguard 323 and Cyabsorb UV-416.

Inorganic, organic and hybrid polymer-based fillers can also be included. Blends containing at least inorganic-organic hybrid polymers and fillers are also referred to as composites. The starting component with cycloolefinic groups and the thiol with at least two thiol groups per molecule as well as any additives dissolved therein act as a matrix in which the fillers are dispersed.

The fillers can be primary particles in the nanometer range, which, as the invention explains further below, can be agglomerated or (completely) dispersed to larger particles as required and show surprising effects, such as the modification of the refractive index of the matrix system. Instead or additionally larger particles can be added, e.g. to modify the mechanical or tribological properties. Inorganic splintered or spherical fillers are preferred, but fibrous fillers can also be used. Dental glass powders with particle sizes between 0.18 μm and 5 μm are particularly preferred as splinter-shaped fillers. As spherical fillers, SiO₂, TiO₂, ZnO, ZnS and ZrO₂ nanoparticles with particle sizes between 5 nm and 100 nm are particularly preferred. Examples of fillers that can be used are also described in EP 3 090 722 A1 (see pages 9 and 10) as well as in DE 1964378, DE 10018405, DE 102011053865 A1 and DE 102005061965.7. To improve the bond between the filler particles and the crosslinked matrix, the fillers may be surface modified. Preferably, the surface modification carries groups which can be polymerized into the organic network of the silica polycondensate. An example is the surface modification with norbornenyl groups or methacrylic groups, e.g. in the form of methacryloxypropyltrimethoxysilane, see EP 3 090 722 A1 (p. 10), if the polysiloxane component of the liquid or viscous material on which the process according to the invention is based has norbornenyl groups, methacrylic groups or other groups copolymerizable with the said groups as organically polymerizable radicals. The surface modification causes an improved dispersion of the particles. If the particles are nanoparticles (below a certain particle size), they no longer scatter light but modify the refractive index of the matrix of which they have become part. The mechanical values of the material are generally only slightly affected by this measure, as can be seen from the examples below. The matrix system with an increased refractive index can then be combined with suitable fillers with a similar refractive index in order to achieve improved mechanical properties while retaining the translucency.

Certain particles such as ZrO₂ and TiO₂ have the additional ability to absorb energy in the form of light, e.g. by raising an electron from the valence band to the conduction band (formation of an electron-hole pair). Subsequently, e.g. recombination takes place again. Thus they also act as light absorbers. In this respect, it is possible to refer to G. Wypych, Handbook of UV Degradation and Stabilization, ChemTec Publishing, Toronto 2015, p. 45. There, the effect is demonstrated using TiO₂ and ZnO nanoparticles.

The surprising thermomechanical effect observed after hardening is shown by the fact that the modulus of elasticity of the shaped body or workpiece drops sharply when the temperature rises. This effect depends on the material used and often occurs within a narrow temperature range (approx. 10 to 40° C., sometimes already approx. 5 to 15 or 20° C., but also significantly higher depending on the starting materials used). Below and above this range, relatively stable, i.e. constant module values are observed. In other words: the photochemically polymerized material changes its consistency from stiff to soft/flexible. The temperature at which the flexibilisation takes place can be adjusted within a wide temperature range by selecting the starting substances and thus adapted to various applications such as the earmoulds described (in the range around body temperature). The C═C and SH content per basic structural unit, the distance of the C═C and SH groups to the core structure and the respective distance to each other, the possible proportion of the Si—O—Si═-based polymer structure and thus the organic, inorganic and total cross-linking density can be used as variables. An important influencing factor is also the degree of conversion during thiol-ene polyaddition. It should be noted here that nobornenyl groups which remain unreacted are unproblematic from an application-specific point of view, as they are non-reactive and thus stable under “normal” conditions. In concrete terms, the following data on the stiffening/flexibilization temperature (“T_{W}”) were determined on exemplary shaped bodies produced according to the invention:

SystemE->Depending on additives/after-treatment in the range≈36-49° C.

System A->Depending on additives/after-treatment in the range≈15-18.5° C.

SystemB->Depending on additives/after-treatment in the range of≈35-50° C.

The dependence on additives can be explained as follows: if the initiator quantity is increased, the degree of crosslinking generally increases, which leads to a higher memory module E′; if, on the other hand, light absorbers are added, the memory module E′ drops slightly.

Other thermal effects observed on the materials of the invention are a reduction in refractive index and an increase in the coefficient of thermal expansion with increasing temperature.

Preferred after-treatments after the actual printing process include post-curing to increase the conversion of the polymerizable groups of the shaped parts. Post-curing can be carried out in an advantageous way photoinitiated with a flashlight unit. In the case of the thiol-ene-polyaddition used according to the invention, an inert gas atmosphere is not necessary to avoid an oxygen inhibition layer. Alternatively or additionally, the light post-curing can be carried out with a Spectramat device (a dental light furnace, Ivolcar, Schaan, (Lichtenstein)) and/or a common dental radiator. Additionally or alternatively, post-curing can be thermally initiated in an oven at elevated temperature (e.g. 100° C.) and/or by means of an IR radiator and/or microwave.

Especially in case of planned thermal post-curing, for example, a photoinitiator can be combined with a thermal initiator, whereby the photoinitiator is used during pre-curing during 3D printing and the thermal initiator is used afterwards during post-curing in the oven. If necessary, a combination of different photoinitiators can also be used, which have their absorption maximum at different wavelengths, whereby one photoinitiator is used for splitting during 3D printing and the second for post-curing with light of a specific wavelength that differs from the emission wavelength of the printer's LED. An example of such a combination is the use of LTPO as an initiator for 3D printing (effective from about 420/410 nm and below) and camphor quinone, effective in a wavelength range of about 400-500 nm, for post-curing. Alternatively, a UV initiator can be used as a second initiator for post-curing. Post-curing with light can be photoinitiated, e.g. with a flashlight unit (optionally under inert gas) and/or Spectramat and/or dental radiator and/or high-intensity LED spotlight (Bluepoint LED eco from Hönle, Gräfelfing/Munich).

The cured materials are characterized by the so-called memory effect and can therefore be used for “deployable” stents, for example. The stent is “folded” or “compressed” at an elevated temperature and thus brought into a modified shape. It is then cooled down to the storage temperature (e.g. in the refrigerator). This stabilizes the changed shape. In case of use, i.e. during an operation, the stent is inserted into the human body in the area of e.g. a stenosis, heated to body temperature within a short time and simultaneously unfolded. The stent thus takes on its original shape and thus expands the narrowed area in a very simple way, allowing unimpeded blood flow.

After the addition of various types of additives, as far as required e.g. for thiol-en-stabilization, initiation, etc., the photosensitive flowable resin systems or composites can be converted (“printed”), according to the invention, with the help of a 3D printing process and especially preferably by means of Digital Light Processing (DLP), into shaped bodies, especially patient-specific components, in which the above-mentioned thermomechanical property profile is combined with high dimensional accuracy/accuracy of shape/accuracy of dimension/dimensionality.

Despite high elasticity/elongation, the systems can be printed well (i.e. without printing errors, such as step formation) (even without support structures/supports). The following problem often arises when printing elastic materials using DLP processes: The pull-off forces of the hardened layer from the bottom of the bath are often very high with flexible materials. This can cause the layer to stick to the bottom of the bath and the already printed component to deform/stretch during the lifting of the building platform, as it does not tear off the bottom. This can cause printing errors, such as undesired steps in the component. To avoid these printing problems, support structures must often be used to support the flexible material. The inventors of the present invention were able to establish that this problem does not exist in the present case.

To achieve an attractive appearance, the shaped bodies/components can be made transparent. Alternatively, fillers or pigments can be added to the material to be polymerised to produce shaped bodies/components with different colour variants (e.g. skin colour).

If the process according to the invention is to be used for the production of earmoulds, the skin compatibility (anti-allergenic behaviour) required for this is also given, since the material which can be used according to the invention does not require the addition of otherwise common (meth)acrylate-based monomers.

To sum up, the following can be said:

An important advantage of the procedure according to the invention can be seen in the fact that a widely investigated and known material basis can be used for e.g. an earmould which, compared to previous materials, is both biocompatible (especially anti-allergenic) and—surprisingly—has the necessary thermo effect for an optimal adaptation to the ear at body temperature and thus a high wearing comfort and, in contrast to previous thermoformable materials, is printable. Thus, a great variability regarding the additional functionalisation and thus the resulting properties (e.g. for the realisation of an antibacterial design) can be resorted to.

Likewise, a location-dependent stiffening (e.g. in the “lid area”) as described above is also possible. This ensures a material-saving, automatable and thus fast and cost-effective process for the production of medical components such as earmoulds. In addition, transparent as well as different colour variants are possible with regard to appealing aesthetics. These can be obtained, for example, by adding coloured inorganic particles such as those containing iron oxide or (usually organic) dyes that are soluble in the liquid or viscous material. Furthermore, the curing process described above is usually a very fast reaction, which allows a reduction of the printing and post-curing time even for very complex components. It is also possible to guarantee the time required for the printing process and more the required (storage) stability of the base materials. The addition of a thiol-ene stabiliser such as pyrogallol still has no negative effects on the printing qualities.

The thermomechanical effect discovered for the first time by the inventors and its adjustable perimeter/adjustable dimension depending on the starting materials used in each case (see also the examples below) enables the cost-effective production (by means of 3D printing process) of patient-specific components such as an earmould, which is not perceived as a disturbing foreign body in the ear, even when in the ear canal for several hours a day. Depending on the nature and sensitivity of the ear canal, adapted, i.e. patient-specific harder or softer systems can be used. Furthermore, the material can be selected to adapt to the ear, i.e. to be hard at room temperature and flexible at body temperature. A material that is firm and grippy at room temperature for good handling can thus be easily inserted and become pleasantly soft at body temperature, thus sealing and preventing pressure pain. At the same time a good basic stability is given for a long durability of the earmould. A high wearing and handling comfort as one of the most important properties of earmoulds can thus be realised.

Especially preferred is the use in the field of hearing acoustics/audio and hearing protection such as
Earpieces for behind-the-ear hearing aids (earmoulds)
Earpieces/shells/housings for in-the-ear hearing aids
Receiver-in-the-ear hearing aids (RITE)
Receiver-In-The-Ear-Canal hearing aids (RIC)
Monitoring (for e.g. musicians, racing drivers, reporters, security forces)
In-Ear Speakers
Hunters, military, pilots, etc. (on the one hand very sensitive hearing and on the other hand reduction of sensitivity when shooting noises)
Hearing protection (e.g. for disco, work with noise (required from 85 dB, e.g. road construction, airport))
Snoring
As protection against water, dirt, etc.++eardrum perforation (tear, hole), carrier of “tympanic tube” (drainage)
Jewellery earmoulds (colourful+rhinestones, unusual shape).

Furthermore, it can be used in the medical sector in general, e.g. for the following applications
Implants→Thin foils can be handled much better at ambient temperature, i.e. in the stiffened form within the scope of further application-related modifications (e.g. application of conductor tracks, etc.) (also already during foil production and post-curing), while during/after implantation they become very flexible, e.g. in brain areas, other soft tissue areas, etc., and thus tissue damage can be avoided.
Artificial blood vessels or stents→It is possible to produce very filigree “hollow fibres” or stents, which are much easier to handle and also to insert at ambient temperature, i.e. in the stiffened form (even during their manufacture and post-curing), while after implantation they become very flexible and adapt to the ambient conditions (e.g. during body movements).
Catheters, needles and the like for infusion systems→For such applications, (highly) filled systems with nanoparticles are preferred. The combination with nanoparticles has the effect, on the one hand, that the shaped bodies produced remain transparent, while on the other hand they have a good hardness or sharpness, so that they can be “pierced” (without separate enveloping needle system) into the skin of the human body, e.g. in the form of infusion needles, at ambient temperature, i.e. in the stiffened form, but become very flexible after contact with the body and the corresponding temperature adjustment, and as an “infusion cannula” adapt to environmental conditions (e.g. during body movements).
(Hard) dimensionally stable (special) contact lenses, e.g. for individual visual defects and eye diseases (mainly individual production with regard to individual corneal geometry), are easier to handle in a stiffened form before insertion (e.g. during care, cleaning, insertion) and adapt to the shape of the bulb after heating to the temperature of the cornea. This results in a high wearing comfort (no/lower foreign body sensation).

The shaped bodies according to the invention can also be used for biological purposes, namely scaffolds and other carriers or substrates for biological material, in particular cells. Cells require mechanical properties (e.g. modulus of elasticity) very precisely adapted to the respective cell type in order to feel comfortable and grow on a substrate. These properties can be easily adjusted with the materials of the invention.

In addition, scaffolds and similar carriers made of materials that can be used according to the invention can be used to create novel effects: Due to their thermomechanical flexibility, it is possible to cause a substantial softening/flexibilization of the scaffolds/carriers by only a small temperature increase above the other, usual ambient temperature of the cells and the (growth) medium in which they are located. By reducing the modulus of elasticity required for the “well-being” of the cells, the cells lose the feeling of being in “familiar” surroundings, they reduce their adhesion to the substrate and can be more easily stripped off. This effect can also be achieved the other way round, by lowering the temperature, whereby the material according to the invention then achieves a modulus of elasticity or hardness that is unfavourable for cell growth and for the adhesion of the cells.

Instead, there are other ways to detach cells that grow on a scaffold or other substrate and adhere well to it. In this case, a material is used as the material usable according to the invention which, in addition to the cycloolefinic groups, also has groups accessible to a ROMP, as explained above. For the scaffold on which the cells are to grow, the material is selected in such a way that it has the required mechanical properties without additional cross-linking by ROMP. However, it contains the ROMP catalyst mentioned above. If the cells are now to be detached, the scaffold/substrate is exposed to the appropriate wavelength at which ROMP is triggered. The additional cross-linking that occurs during this process stiffens the material and the cells can be detached more easily.

Furthermore, the use in the following areas is possible
(Micro)optics and (Micro)electronics Principle→see above for implants
Optical, electronic and mechanical switches

Curing by the thiol-ene reaction can take place via two different mechanisms: A radical thiol-ene reaction with all C═C double bonds, e.g. with C═C double bonds of the norbornenyl group, is possible, whereas a thiol-Michael addition only occurs with activatable C═C double bonds, as they may be present, e.g. in the form of methacrylic groups, especially methacrlyate. The radical mechanism is initiated by a radical initiator (thermal and/or photoinduced and/or redox-induced). Especially non-activated C═C double bonds such as the double bond of the norbornenyl group are suitable for this type of initiation. In order to be able to initiate also a thiol-Michael addition locally and/or temporally targeted, photobases can be used, since the thiol-Michael addition is base-catalyzed. Photobases release a base upon exposure to light and can thus initiate the Thiol-Michael addition. The preparation and use of photobases is described in the literature.

If photochemical work is performed, irradiation can be carried out with visible and/or UV light. Combinations of different conversions, for example photochemical and thermal or a combination of redox-induced with e.g. photo induced or thermal curing are also possible.

In the following, the invention will be explained in more detail by means of concrete examples of implementation, whereby these must of course not be regarded as restrictive.

A. Syntheses

I. Synthesis of Resin System-1 (Silane-Based System)

1. Synthesis Stage (from DE 4416857) (Preparation of a Methacrylate-Based Silane Resin with Free OH Groups

Base Resin (a):

Triphenylphosphine as catalyst, BHT as stabiliser and then 47.35 g (0.550 mol) of methacrylic acid are added dropwise to 125.0 g (0.503 mol) of 3-glycidyloxypropymethyldiethoxysilane under a dry atmosphere and stirred at 80° C. (for about 24 hours). After addition of acetic ester (1000 ml/mol silane) and H₂O for hydrolysis with HCl as catalyst, stirring is carried out at 30° C. After stirring for approx. several days, processing is carried out by repeated shaking out with aqueous NaOH and with water and filtration through hydrophobic filters. It is first rotated off and then removed with oil pump vacuum. The result is a liquid resin without the use of reactive diluents (monomers) with a viscosity of approx. 3-5 Pa·s at 25° C.

2. Synthesis Stage (from DE 102011054440A1) (Conversion of Methacrylate Groups with Dicyclopentadiene to Norbornenyl Groups, Resin System-1)

About 45.5 g (0.69 mol) cyclopentadiene (CP) (freshly prepared by cleavage of dicyclopentadiene) is distilled to 80.0 g (0.30 mol) of base resin (a) while stirring at about 90° C., and then stirring is continued for about 1-2 h at 90° C. The reaction can be followed by NMR. The volatile components such as unreacted cyclopentadiene are removed in an oil pump vacuum at temperatures up to 90° C. The result is a liquid resin with a viscosity of approx. 53-110 Pa·s at 25° C. Further processing is usually not necessary.

II. Synthesis of Resin System-2a/b/c (Silane-Based System)

1. Synthesis Stage (from DE 103.49766.8) (Producing a Silane Resin Having Partly One Methacrylic Group Per Silicon Atom, Partly One Methacrylic and One Acrylic Group Per Silicon Atom, the Methacrylic and Acrylic Groups Each being Present on the Same Radical Bonded to the Silicon Via Carbon, by Reacting the Free OH Group with Acylic Acid Chloride)

Base Resin (b₁) (Ratio of Methacrylic to Acrylic Groups 1:0.74)

36.66 g (0.405 mol) acrylic acid chloride is added dropwise to 120.7 g (0.45 mol) base resin (a) (from I.) and 45.1 g triethylamine (0.446 mol) in 450 ml THF as solvent under a dry atmosphere and cooling is carried out by means of an ice bath while stirring and continued at RT. After the usual processing to separate the amine hydrochloride and acidic by-products formed during the addition and removal of the volatile components with oil pump vacuum, a liquid resin with a viscosity of about 2.7 Pa·s at 25° C. is obtained.

Base Resin (b₂) (Ratio of Methacrylic to Acrylic Groups 1:0.30):

In accordance with the instructions in relation to base resin (b₁), 78.15 g (0.30 mol) of base resin (a) (from I.) and 11.69 g of triethylamine (0.385 mol) in 300 ml of THF are reacted with acrylic acid chloride (9.50 g (0.105 mol)), the resulting reaction mixture is further processed and worked up. The result is a liquid resin with a viscosity of about 2.7 Pa·s at 25° C.

Base Resin (b₃) (Ratio of Methacrylic to Acrylic Groups 1:0.45):

In accordance with the instructions in relation to base resin (b₁), 78.15 g (0.30 mol) of base resin (a) (from I.) and 16.70 g of triethylamine (0.55 mol) in 300 ml of THF are reacted with acrylic acid chloride (13.52 g (0.15 mol)), the resulting reaction mixture is further processed and worked up. The result is a liquid resin with a viscosity of about 2.5 Pa·s at 25° C.

2. Synthesis Stage (from DE 102011054440A1) (Conversion of Methacrylate and Acrylate Groups with Dicyclopentadiene to Norbornenyl Groups)

Resin System-2a (Ratio 1.74 Norbornenyl Groups Per Silicon Atom):

About 22.0 g CP are added dropwise to 92.4 g of base resin (b₁) while stirring at about 50° C. After heating the reaction mixture to approx. 90° C., 39.2 g CP are added while stirring and then stirred for approx. 1.5 h at 90° C. The volatile components such as unreacted cyclopentadiene are removed in an oil pump vacuum at temperatures up to 90° C. The result is a liquid resin with a viscosity of approx. 380 Pa·s at 25° C. Further processing is usually not necessary.

Resin System-2b (Ratio 1.30 Norbornenyl Groups Per Silicon Atom):

CP is added to 63.7 g of base resin (b2) in accordance with the instructions in relation to resin system 2a (a total of approx. 38.1 g), thus converted and the resulting reaction mixture is worked up. The result is a liquid resin with a viscosity of about 177 Pa·s at 25° C. Further processing is usually not necessary.

Resin System-2c (Ratio 1.45 Norbornenyl Groups Per Silicon Atom):

CP is added to 63.7 g of base resin (b3), in accordance with the instructions in relation to resin system 2a (a total of approx. 77.7 g), thus converted and the resulting reaction mixture worked up. The result is a liquid resin with a viscosity of about 194 Pa·s at 25° C. Further processing is usually not necessary.

Note: The viscosities of resins (b1) to (b3) and of resin systems-2a, 2b and 2c are highly dependent on the precise synthesis and processing conditions selected in each case, especially for the precursors. The values may therefore deviate in the case of reworking.

III. Synthesis of Resin System-3 (Purely Organic System, Conversion of Glycerine Dimethacrylate to the Corresponding Norbornenyl Derivative)

Resin System-3

About 39.7 g CP are added dropwise to 30.00 g (0.131 mol) of glycerol dimethacrylate (isomer mixture) with dissolved 0.04% by weight butyl hydroxytoluene (BHT) while stirring at about 80° C., and stirring is continued. The conversion of the methacrylate group is monitored by ¹H-NMR. The volatile components such as unreacted cyclopentadiene are removed under vacuum at temperatures up to 90° C. The result is a liquid resin with a viscosity of approx. 6.3 Pa·s at 25° C. Further processing is usually not necessary.

B. Production of Resin Blends for 3D Printing and Compared to Conventional Processing

General instruction: In the respective resin system, a light stabilizer may be dissolved in advance, for which a solvent may be added, which is then removed again. Subsequently, the thiol, in which a photoinitiator and, if a strained ring system is used as cycloolefinic group, a thiol-ene stabilizer was previously dissolved under stirring at elevated temperature (usually approx. 40° C.), is added. The resulting resin blends are used for 3D printing and for classic test specimen production in the context of a preferably photo-induced thiol-ene polyaddition.

As resin systems the above-mentioned systems 1, 2a, 2b, 2c and 3 were used.

The optical brightener 2,5-bis(5-tert-butyl-benzoxazol-2-yl)thiophene (TBT) served as an example for a light stabilizer, which was used here at 0.0003-0.0005 mmol/g resin mixture (based on resin system+thiol).

Unless otherwise specified, the thiol used was the trithiol trimethylolpropanetri(3-mercaptopropionate) (TMPMP) (usually in a molar ratio of SH:C═C=0.9:1), in which the stabilizer pyrogallol (0.05-0.2 wt. % based on resin system+thiol) and the photoinitiator LTPO (1.0 or 2.0 wt. % based on resin system+thiol) had been dissolved while stirring at 40° C. In a further case, a mixture consisting of TMPMP and 2,3-di((2-mercaptoethyl)thiol-1-propanethiol (DMPT; it contains no ester groups but additional thioether groups which increase the refractive index) in a molar ratio of 0.3:0.9 served as thiol. The molar ratio of SH:C═C is 0.9:1. Otherwise, everything remained the same.

Pyrogallol was used as a thiol-ene stabiliser in this specific case.

C. Production of Composites for 3D Printing and in Comparison with Conventional Processing

C1. General Instruction for Composite Production with TiO₂ as Filler:

0.5 wt. % TiO₂ filler (in agglomerated form; rutile; primary particle 0=10-30 nm; 99.5%; from io-li-tec nanomaterials) is incorporated in the respective resin mixture (from B., with a molar ratio of SH:C═C=0.9:1) in the speed mixer (2×60 sec). The materials are then evacuated at 37° C. until they are bubble-free. The resulting composites are used for 3D printing and for classic test specimen production in the context of a preferably photo-induced thiol-ene polyaddition.

C2. General Instruction for Composite Production with ZrO₂—NP as Filler:

A dispersion of ZrO₂—NP from Pixelligent (PixClear PCPB-2-50-ETA, 0=7-10 nm, 50 wt. % dispersed in ethyl acetate, methacrylate functionalised) is added to the respective resin mixture (from B.), and the material is stirred at RT. The next day the solvent is removed by vacuum at 40° C. The resulting composites with a ZrO₂ content of 30 wt. % are used for 3D printing and for classic test specimen production in the context of a preferably photo-induced thiol-ene polyaddition.

D. Determination of the Refractive Index Change and Mechanical Data

D1. Test Specimen Production for the Determination of the Refractive Index Change in the T_{W} Range:

The resin mixture consisting of resin system 2b and 1% LTPO (based on resin system 2b+thiol) and thiol TMPMP (in a molar ratio of SH:C═C=0.9:1) and stabilizer pyrogallol (0.2% by weight based on resin system 2b+thiol) is placed in a mould (20×8×4 mm³). By means of dental lamps (Polofil Lux of Voco GmbH, Cuxhaven); (each 100 sec VS/RS) and flash unit G171Otoflash (NK Optik, Baierbrunn, D) (3000 flashes) the C═C double bonds of the norbornenyl groups are converted in a photo-induced polyaddition and the resin mixture is cured. The refractive index before or after heating is determined by means of an Abbe refractometer (immersion liquid is 1-bromonaphthalene) and thus the change in refractive index in the T_{W} range. As the heating was done with a hair dryer, as it was not possible to temper the sample during the measurement, the values are not very accurate.
Refractive index before heating (at 20° C.)≈1.548
Refractive index after heating (approx. 40-60° C.)≈1.533

Result: Δn≈0.015 ⇒The refractive index decreases as the samples become more flexible and thus probably the structural density decreases.

D2. Classic Test Specimen Production to Determine the Mechanical Data (Abbreviated as “Classic” in the Table):

The respective resin mixture (made of B.) or the composite (C1. or C2.) is placed in a rod form (40×2×5 mm³). Using dental radiators (Polofil Lux; 100 sec each VS/RS), the C═C double bonds of the norbornene groups are preferably converted in a photoinduced polyaddition and the resin mixtures or composites are cured.

D3. Test Specimen Production to Determine the Mechanical Data Using 3D Printing (Abbreviated as “3D Printing” in the Table):

The rod-shaped test specimens (40×2×5 mm³) are printed on a Rapidshape S 60 LED 3D printer (DLP principle) with a layer thickness of 100 μm and an exposure time of 31 s per layer at a light intensity of approx. 6.15 W/m² with perpendicular orientation to the building platform (corresponds to 40 mm in z-direction), then washed in isopropanol for 90 s in an ultrasonic bath, blown off with compressed air, dried for 5 min at RT and then post-cured by means of the UV lamp Bluepoint LED eco (200 sec. in total), flash unit G171Otoflash (3000 flashes) or Spektramat (3 min).

The DMA (dynamic mechanical analysis) tests on the above specimens were performed using NETZSCH DMA242C with the following settings: Bearing distance=20 mm; frequency=1 Hz; heating rate=2.0° C./min; max. dynamic force=4.00 N; atmosphere=air. The memory module E′_{RT} (at 22° C.), the minimum memory module E′ₘᵢₙ and the temperature T_{W} (at 2·E′ₘᵢₙ) were determined, i.e. the temperature at which the material has almost the most flexible consistency (“almost” because E′ converges towards E′ₘᵢₙ as the temperature increases. Strong increases in temperature, which are irrelevant for the application, therefore lower E′ only insignificantly above 2·E′ₘᵢₙ). The data are given in the table below; the DMA curves are shown in FIG. 1.

| | | | DMA data | | | |
|---|---|---|---|---|---|---|
| Resin mixture/composites | | | Test piece | E′_{RT} | E′ₘᵢₙ | T_{W} |
| System | Composition | Additives | production | [MPa] | [MPa] | [° C.] |

Resin blends (unfilled)

| | | | | | | |
|---|---|---|---|---|---|---|
| A_{K} | Resin system 1 + | 0.02% Pyrogallol/ | Classic | n.d. | n.d. | 18.5 |
| | thiol | 1% LTPO | | | | |
| A_{D} | Resin system 1 + | 0.2% Pyrogallol/ | Printed + | 111 | 99 | 15.0 |
| | thiol | 1% LTPO/ | flash device | | | |
| | | 0.0005 mmol/g TBT | | | | |
| BK₁ | Resin system 2a + | 1% LTPO | Classic | 2550 | 30 | 50 |
| | thiol | | | | | |
| BK₂ | Resin system 2a + | 0.02% Pyrogallol/ | Classic | 1550 | 34 | 35 |
| | thiol | 1% LTPO | | | | |

Viscosity at 25° C./40° C. → B_{K1} ≈ 16/4.5 Pa · s B_{K2} ≈ 8.2/2.3 Pa · s

| | | | | | | |
|---|---|---|---|---|---|---|
| C_{K} | Resin system 2b + | 1% LTPO | Classic | 1370 | 19 | 41 |
| | thiol | | | | | |
| C_{D} | Resin system 2b + | 0.2% Pyrogallol/ | Printed + | 678 | 53 | 32 |
| | thiol | 1% LTPO/ | flash device | | | |
| | | 0.0005 mmol/g TBT | | | | |
| E_{K1} | Resin system 2c + | 1% LTPO | Classic | 1880 | 27 | 45 |
| | thiol | | | | | |
| E_{K2} | Resin system 2c + | 0.05% Pyrogallol/ | Classic | 1920 | 24 | 49 |
| | thiol | 2% LTPO | | | | |
| E_{D1} | Resin system 2c + | 0.05% Pyrogallol/ | Printed + | 1630 | 33 | 41 |
| | thiol | 1.5% LTPO | Spectra- | | | |
| | | | mat | | | |
| E_{D2} | Resin system 2c + | 0.2% Pyrogallol/ | Printed + | 1000 | 51 | 36 |
| | thiol | 1% LTPO/ | flash device | | | |
| | | 0.0005 mmol/g TBT | | | | |
| E_{D3} | Resin system 2c + | 0.2% Pyrogallol/ | Printed + | 1437 | 15 | 47 |
| | thiols | 1% LTPO | flash device | | | |
| | TMPMP/DMPT | | | | | |
| D_{K} | Resin system 3 + | 1% LTPO | Classic | 1640 | 10 | 45 |
| | Thiol | | | | | |

Composites

| | | | | | | |
|---|---|---|---|---|---|---|
| F_{D} | Resin system 1 + | 0.2% Pyrogallo/ | Printed + | 130 | 26 | 27 |
| | thiol + | 1% LTPO | flash device | | | |
| | 30% ZrO₂—NP | | | | | |

The results show that, depending on the resin structure (e.g. C═C number per structural unit, i.e. the organic cross-linking density), the additives used or their concentration (which can vary to meet the respective requirements with regard to the cross-linking density), the additives used or their concentration (which can be different to meet the respective requirements with regard to the cross-linking density), the resin structure and the cross-linking density can be adjusted. the desired printing result, the resolution, the complexity of the structures and the like), the memory module E′_{RT}, the memory module E′ₘᵢₙ as well as the temperature T_{W} (at 2·E′ₘᵢₙ) and in particular also the change/the extent of the stiffness/flexibility in the range around the temperature at E′ₘᵢₙ (↔thermal effect) can be adjusted within wide limits for different applications.

For example, the table above shows the following:

The comparison of A_{K} and A_{D} shows that TBT (and therefore probably other light stabilizers as well) has hardly any effect on the temperature T_{W}. In some cases, as already noted above, it decreases slightly.

With increasing number of norbornenyl groups per silicon atom and thus with increasing crosslinking (because the ratio SH to C═C double bond remains the same) the temperature T_{W} increases: For A_{D} with one norbornenyl group per silicon it is 15.0° C., for C_{D} (1.30 norbornenyl groups per silicon atom) it is 32° C., and for E_{O2} (1.45 norbornenyl groups per silicon atom) it is 36° C. This shows that the temperature T_{W} can be adjusted within wide limits and that higher values than those shown in the examples can be achieved. The value can also be further increased by adding suitable quantities of suitable fillers. In this way, values for T_{W} of more than 50° C. should be achievable, e.g. up to 60° C., up to 70° C., up to 80° C., up to 90° C. or even up to 110° C.

The addition of the nanoparticles of ZrO₂ results in a transparent composite (see FD in the table above) and, as expected, the memory module and, surprisingly, the temperature at E′ₘᵢₙ increases compared to the unfilled resin mixture A_{K}. This also allows the use of fillers to increase the flexibilization temperature (↔thermal effect) and thus shift the range of application to higher T-ranges.

The inventors were also able to show that when dispersed (isolated!) inorganic, preferably oxidic nanoparticles (especially in the size range of the primary particles of approx. 5-100 nm) (presently used in an amount of 30 wt. %, see FD) are added to the product, the addition of a light absorber such as TBT can be dispensed with for the production of shaped bodies with overhangs or boreholes, as they can themselves act as photoabsorbers. Furthermore, the addition of these particles increases the refractive index of the matrix of which they have become a part, as explained above.

With increasing number of norbornenyl groups per silicon atom and thus with increasing cross-linking (because the ratio SH to C═C double bond remains the same) E′_{RT} increases: For C_{K} with 1.30 norbornenyl groups per silicon atom it is 1370 MPa, for E_{K1} (1.45 norbornenyl groups per silicon atom) it is 1880 MPa, and for B_{K1} (1.74 norbornenyl groups per silicon atom) it is 2550 MPa. This shows that the memory module E′_{RT} can be adjusted within wide limits at room temperature.

The same applies to E′_{RT} for the following systems: For A_{D} with one norbornenyl group per silicon, E′_{RT} is 111 MPa, for C_{D} (1.30 norbornenyl groups per silicon atom) it is 678 MPa, and for E_{D2} (1.45 norbornenyl groups per silicon atom) it is 1000 MPa.

This also shows that the memory module E′_{RT} is adjustable within wide limits at room temperature.

With increasing number of norbornenyl groups per silicon atom and thus with increasing cross-linking (because the ratio SH to C═C double bond remains the same) E′ₘᵢₙ increases: For C_{K} with 1.30 norbornenyl group per silicon it is 19 MPa, for E_{K1} (1.45 norbornenyl groups per silicon atom) it is 27 MPa, and for B_{K1} (1.74 norbornenyl groups per silicon atom) it is 30 MPa. This shows that the memory module E′ₘᵢₙ is adjustable within wide limits.

The simultaneous addition of pyrogallol and TBT causes E′_{RT} to decrease and E′ₘᵢₙ to increase. This can be seen in the systems C_{K} (no pyrogallol/TBT) with an E′_{RT} of 1370 MPa and E′ₘᵢₙ of 19 MPa compared to C_{D} (0.2 wt % pyrogallol and 0.0005 mmol/g TBT) with an E′_{RT} of 678 MPa and E′ₘᵢₙ of 53 MPa. The same applies to systems E_{K1} (no pyrogallol/TBT) with an E′_{RT} of 1880 MPa and E′ₘᵢₙ of 27 MPa and E_{D2} (0.2 wt. % pyrogallol and 0.0005 mmol/g TBT) with an E′_{RT} of 1000 MPa and E′ₘᵢₙ of 51 MPa.

By the proportional use of the trithiol DMPT, which has no ester groups but thioether groups and also has a shorter connecting structure between the thiol groups than the trithiol TMPMP, a higher E′_{RT} at lower E′ₘᵢₙ (15 MPa) in combination with a higher temperature T_{W} (47° C.) is achieved in comparison with system E_{D2} with 1437 MPa. Thus, the material characteristics can also be significantly modified by using a thiol with a modified bonding structure between the thiol groups.

The coefficient of thermal expansion of the conventionally and inventionally produced shaped bodies should increase very strongly in the T_{W} range, as it generally correlates with the flexibility of the shaped bodies. In addition, the refractive index decreases with increasing temperature, as explained above. Therefore, the shaped parts should also be suitable for use as optical or mechanical switches.

E. 3D Printing Production of Modelotoplastics (Earmoulds)

The earmoulds corresponding to the model shown in FIG. 4 were printed with the unfilled material systems C_{D} and E_{D2} using the 3D printer S 60 LED from Rapidshape (DLP principle) with a layer thickness of 100 μm and an exposure time of 31 s per layer at a light intensity of 6.15 W/m² with the orientation to the building platform as shown in the figure, then washed in isopropanol for 3 min in an ultrasonic bath, blown through/blown off with compressed air, dried for 5 min at RT and dried by means of a UV lamp (in total, the earmoulds were then placed in a vacuum bath for 3 min. 200 sec.), flash unit (3000 flashes) or Spektramat (3 min).

The printed earmoulds are transparent, have a good surface quality and contain well resolved through holes. At room temperature they are solid/little flexible and at body temperature they show a soft/flexible behaviour. The earmoulds based on E_{D2} at RT and at body temperature show a higher stiffness than those based on C_{D}.

Thus, earmoulds of high quality in combination with different thermo-/E-module behaviour and thus patient-specific requirements can be produced by means of 3D printing.

F. 3D Printing Production of “Hose-Like” Shapes

With the unfilled material system E_{D2}, tube-like shaped parts were produced as a model for infusion needles as shown in FIG. 5, artificial blood vessels, catheters were printed with the 3D printer S 60 LED from Rapidshape (DLP principle) with a layer thickness of 100 μm and an exposure time of 31 s per layer at a light intensity of 6.15 W/m² printed with vertical alignment to the building platform, then washed in isopropanol for 3-4 min in an ultrasonic bath, blown through/blown off with compressed air, dried for 5 min at RT and dried by means of a UV lamp (in total 200 sec.) or flash unit (3000 flashes).

They are transparent, have a good surface quality (inside/outside) with a well formed “tip” (example infusion needle) with an outer diameter of 2.0 or 3.0 mm and a wall thickness of 0.5 or 0.75 mm. At room temperature they are less flexible and at body temperature they show a soft/flexible behaviour.

Thus, tube-like medical shaped parts of high quality in connection with different diameters/wall thicknesses and thus individual requirements can be produced using 3D printing.

G. Coefficient of Thermal Expansion

A rod-shaped test specimen (25 mm×2 mm×2 mm) based on system E_{K3} (corresponding to example D) with the following parameters was produced: Composition: resin system 2c+thiol (TMPMP); additives: 0.2% pyrogallol/1% LTPO; test piece production: Classic+5 min (3000 flashes) with the flash device.

The temperature T_{W} was about 36° C.

The temperature-dependent expansion coefficient α is determined with the dilatometer from Netzsch (DiL 402C) at a heating rate of 2K/min. The following values were obtained:
α=90-10⁻⁶ K⁻¹ in the T-range from 24-28° C.
α=101-10⁻⁶ K⁻¹ in the T-range from 24-30° C.
α=163-10⁻⁶ K⁻¹ in the T-range from 26-38° C.
α=248-10⁻⁶ K⁻¹ in the T-range from 40-80° C.

A very strong increase of the α-value with temperature was observed, especially in the range of the T_{W}-value.

H. Memory Effect

Rod-shaped test specimens (2 mm×5 mm×40 mm) based on the E_{K1} system described in example D are used.

The samples are heated to 40° C. in a drying oven, bent by clamping them in a glass (see FIG. 6A), cooled to 13 to 14° C. in the refrigerator and then stored exposed for 1 day at 13 to 14° C. During this process, the bend is maintained according to the clamped shape. The bent specimens are then heated to 37° C. in the drying oven, where they return to their original linear rod shape within about 30 minutes (see FIG. 6B).

1. A process for producing a shaped body with the aid of a radiation-induced printing process, characterized in that
the shaped body is produced by solidification of a liquid or viscous material containing a starting component with cycloolefinic groups, a thiol with at least two thiol groups per molecule and an initiator and/or catalyst for the radiation-induced thiol-ene-addition reaction between the thiol groups and a double bond of the cycloolefinic groups, and
the solidification is effected by directing light from a radiation source onto a region of a surface of a substrate, a layer of the liquid or viscous material located there being subjected to organic polymerisation by the action of radiation and thereby solidified, whereupon further layers of the liquid or viscous material, which are each located on the layer of the most recently solidified material, are successively solidified with the aid of this radiation source,
wherein the shaped body undergoes a drop in its memory module of at least 500 MPa within a temperature window of −25° C. to 110° C. at a temperature rise of at most 20 K, determined by a DMA test on the shaped body by means of NETZSCH DMA242C with the following settings: support distance=20 mm; frequency=1 Hz; heating rate=2.0° C./min; max. dyn. force=4.00 N; atmosphere=air.

2. The process according to claim 1, wherein the liquid or viscous material is located in a bath container having a bottom which is at least partially translucent, and the substrate is a platform immersed in the liquid or viscous material and movable away from the bottom of the bath container.

3. The process according to one of the preceding claims, wherein the starting component with cycloolefinic groups is a silicic acid (hetero)polycondensate modified with mono- or bicycloolefinic groups.

4. The process according to one of the preceding claims, wherein the starting component with cycloolefinic groups is a silicic acid (hetero)polycondensate of or with silanes in which radicals bonded to the silicon via carbon have a norbornenyl group, a norbornenyl group and a hydroxy group or two norbornenyl groups, or wherein the starting component with cycloolefinic groups is a purely organic compound which has two terminal norbornenyl groups.

5. The process according to any of the preceding claims, wherein the thiol having at least two thiol groups per molecule is a trithiol or a mixture of two trithiols.

6. The process according to claim 5, wherein the trithiol is trimethylolpropanetri(3-mercaptopropionate) (TMPMP) and/or 2,3-di((2-mercaptoethyl)-thiol-1-propanethiol (DMPT).

7. The process according to one of the preceding claims, wherein the shaped body is produced in a bath from the liquid or viscous material and is removed from the bath after its formation, washed with a solvent, dried and then post-hardened.

8. The process according to one of the preceding claims, wherein the liquid or viscous material further contains a thiol-ene stabilizer.

9. The process according to one of the preceding claims, wherein the shaped body is suitable for at least one use from the group comprising the use as an earmould, the use for hearing acoustics/audio purposes, the use for hearing protection purposes, the use as an insertion needle, stent, infusion needle and the use as a carrier for cultivating biological organisms or cells.

10. A shaped body obtainable by the process of any of the preceding claims.

11. The shaped body according to claim 10, having been formed in a spatial direction of individual layers having a thickness in the range of 3 to 150 μm or in which the layers have been formed by continuous exposure to light.

12. The shaped body according to claim 10 or 11 having a memory module E′_{RT} (at 22° C.) between 20 and 4000 MPa, a minimum memory module E′ₘᵢₙ between 5 and 300 MPa and a temperature T_{W} (at 2·E′ₘᵢₙ) between 0° C. and 110° C., in each case determined by a DMA test according to the process specified in claim 1.

13. The shaped body according to any of claims 10 to 12, which is an earmould, an injection needle, a stent, or an infusion needle.

14. The use in the medical field of a shaped body according to one of claims 10 to 13, selected from the use as an earmould and/or for hearing acoustics/audio and/or hearing protection purposes or as a piercing or infusion needle or stent or as a carrier for cultivating biological organisms or cells.

15. The use of a shaped body according to any one of claims 10 to 13 as an optical or mechanical switch.
